(19) United States
(12) Patent Application Publication
(10) Pub. No.: US 2026/0144255 A1
(43) Pub. Date: May 28, 2026

(54) ANTIFUNGAL DIFERULIC ACID FORMULATION CONTAINING POACIC ACID AND POACIDIENE, METHOD FOR PRODUCTION, AND METHOD OF USING SAME

(71) Applicant: Wisconsin Alumni Research Foundation, Madison, WI (US)

(72) Inventors: John Ralph, Madison, WI (US); Fachuang Lu, Madison, WI (US); Mehdi Kabbage, Madison, WI (US)

(73) Assignee: Wisconsin Alumni Research Foundation, Madison, WI (US)

(21) Appl. No.: 19/394,334

(22) Filed: Nov. 19, 2025

Related U.S. Application Data
(60) Provisional application No. 63/724,700, filed on Nov. 25, 2024.

(51) Int. Cl. A01N 37/06; A01P 3/00; C07C 51/285; C07C 51/38; C07C 57/03

(52) U.S. Cl. CPC A01N 37/06; A01P 3/00; C07C 51/285; C07C 51/38; C07C 57/03; C07C 2601/10

(57) ABSTRACT

A method to produce a mixture comprising poacic acid (X) and poacidiene (Y) and the resulting product-by-process. The method includes the steps of subjecting a solution of ferulic acid to a free-radical coupling reaction to yield an intermediate mixture comprising ferulic acid dehydrodimers and decarboxylating the intermediate mixture to yield a product mixture containing poacic acid (X) and poacidiene (Y).

### CROSS-REFERENCE TO RELATED APPLICATIONS

Priority is hereby claimed to provisional application Ser. No. 63/724,700, filed Nov. 25, 2024, which is incorporated herein by reference.

### FEDERAL FUNDING STATEMENT

This invention was made with government support under DE-SC0018409 awarded by the US Department of Energy. The government has certain rights in the invention.

### BACKGROUND

Phytopathogenic fungi are responsible for dramatic losses in agricultural production. They infect various parts of plants leading to the destruction of crops. Mycotoxin contamination in food products caused by fungi threatens the security and safety of food and results in huge economic losses. Fungicides have long been applied to manage plant diseases caused by phytopathogenic fungi although their use has been criticized for their many unfavorable side-effects. For instance, continuous use (especially for a single-site antifungal agent) leads to resistance, and excessive use or improper handling of fungicides can have detrimental impacts on people, the environment, and non-target organisms. The highly efficient copper-based fungicides in organic agriculture now face restrictions due to copper accumulation in soils. See Liu, et al., Chemistry & Biodiversity 2020, 17 (12); Makhuvele, et al., Heliyon 2020, 6 (10), e05291: 1-11; Pose, et al., Journal of Agricultural and Food Chemistry 2009, 57 (7), 2843-2848; Mackie, et al., Soil Biology & Biochemistry 2013, 65, 245-253; Wightwick, et al., Journal of Agricultural and Food Chemistry 2010, 58 (1), 449-457; and Wightwick, et al., Environmental Science and Pollution Research 2013, 20 (3), 1574-1585.

New lead compounds for antifungal agents with new mechanisms of action are urgently required. Recently, it was demonstrated that poacic acid (PA), a plant natural product, has antifungal activity against a range of plant pathogens and against Saccharomyces cerevisiae by inhibiting β-1,3-glucan synthesis in fungal cell wall formation. Further investigation suggested that PA and caspofungin are different in the mode of action, although they both target the synthesis of cell wall β-1,3-glucan. See Piotrowski, et al., Proceedings of the National Academy of Sciences of the United States of America 2015, 112 (12), E1490-E1497; Yue, et al. Industrial Crops & Products 2017, 103, 240-243; and Kabbage, et al., Plant Pathology 2020, 69 (1), 112-119; Lee, et al., The Cell Surface 2018, 3, 12-25.

Another diferulic acid derivative, (2E,3E)-4-(4-hydroxy-3-methoxyphenyl)-2-[(4-hydroxy-3-methoxyphenyl)methylidene]but-3-enoic acid, hereafter referred to as poacidiene (Y; FIG. 1), was recently recognized as a potent antifungal agent that exhibited even better antifungal activity than poacic acid. Studies found that both poacic acid and poacidiene inhibit the growth of pathogenic fungi, including filamentous fungi and oomycetes, but through different modes of action. Poacic acid targets cell wall β-1,3-glucan synthase whereas poacidiene affects the DNA-damage-response pathway. Such different mechanisms of action imply that diferulates and their derived compounds have diverse and orthogonal physiological activities. See Ohnuki, et al., Nature Systems Biology and Applications 2022, 8 (1), 3:1-16.

Compared to other widely-used agricultural fungicides, poacic acid and poacidiene, like other botanical fungicides, offer some notable advantages. These plant-derived diferulic acids can be rapidly broken down in the soil whereas the copper-based fungicides will accumulate to toxic levels. (Chen, et al., Applied Microbiology and Biotechnology 2011, 89 (5), 1653-1663. Piotrowski, et al. Proceedings of the National Academy of Sciences of the U.S. Pat. No. 2,015,112 (12), E1490-E1497.) To evaluate the possibility of using mixtures of poacic acid and poacidiene as a fungicidal formulation (as well as to allow other studies on the compounds), far larger quantities of these compounds are required. To meet this need, an improved large-scale synthetic procedure has been developed to produce kilograms of crystalline ethyl ferulate. The ethyl ferulate was used to derive hundreds of grams of crude 8-5-diferulate, which was then decarboxylated and purified by flash chromatography to yield poacic acid (compound X; see FIG. 1) with reasonable overall yields. (Yue, F.; Gao, R.; Piotrowski, J. S.; Kabbage, M.; Lu, F.; Ralph, J., “Scaled-up production of poacic acid, a plant-derived antifungal agent,” Industrial Crops & Products 2017, 103, 240-243.) However, this procedure has several disadvantages. Namely, it uses large volumes of organic solvents (ethanol and acetone), has long reaction times for only a three-step process, and requires costly chromatographic purification.

Ferulate dimerization through free-radical coupling reactions is a straightforward way to synthesize various diferulates. However, separating the individual products is not easy. See Lu, et al., Journal of Agricultural and Food Chemistry 2012, 60 (34), 8272-8277.

Thus, there remains a long-felt and unmet need for a simpler, more straightforward synthetic route to poacic (X) and poacidiene (Y) (see FIG. 1).

### SUMMARY

Disclosed and claimed herein is a method to produce a mixture comprising poacic acid (X) and poacidiene (Y) from ferulic acid (F), the method comprising:
a. subjecting a solution of ferulic acid to a free-radical coupling reaction to yield an intermediate mixture comprising ferulic acid dehydrodimers; and
b. decarboxylating the intermediate mixture of step (a) to yield a product mixture comprising poacic acid (X) and poacidiene (Y).

In preferred versions, steps (a) and (b) are conducted in a single reaction vessel, and steps (a) and (b) are conducted without any intervening processing steps between steps (a) and (b).

In certain versions, step (b) comprises decarboxylating the intermediate mixture of step (a) by heating the intermediate mixture of step (a). In some embodiments, step (b) comprises heating the mixture of step (a) to a temperature of from about 50° C. to about 100° C., for from about 30 minutes to about 3 hours. In some embodiments, step (b) comprising heating the mixture of step (a) to a temperature of about 75° C. for from about 30 minutes to about 2 hours.

The method may further comprise, after step (b):
c. acidifying the product mixture of step (b) and extracting the product mixture with ethyl acetate.

In some embodiments, step (c) comprises acidifying the product mixture of step (b) to a pH of from about 1.0 to about 4.5. In some embodiments, step (c) comprises acidifying the product mixture of step (b) to a pH of from about 1.0 to about 4. In some embodiments, step (c) comprises acidifying the product mixture of step (b) to a pH of from about 1.0 to about 3.

Alternatively, the method may comprise, after step (b):
c. acidifying the product mixture of step (b) to precipitate a mixture comprising poacic acid (X) and poacidiene (Y).

In some embodiments, step (c) comprises acidifying the product mixture of step (b) to a pH of from about 1.0 to about 4.15.

In preferred versions, the solution of step (a) is an aqueous solution.

Also disclosed and claimed herein is a method to produce a mixture comprising poacic acid (X) and poacidiene (Y) from ferulic acid (F), the method comprising:
a. subjecting an aqueous ferulic acid solution to a free-radical coupling reaction to yield an intermediate mixture comprising ferulic acid dehydrodimers; and
b. decarboxylating the intermediate mixture of step (a) to yield a product mixture comprising poacic acid (X) and poacidiene (Y).

In preferred versions, steps (a) and (b) are conducted in a single reaction vessel, and steps (a) and (b) are conducted without any intervening processing steps between steps (a) and (b).

In certain versions, step (b) comprises decarboxylating the intermediate mixture of step (a) by heating the intermediate mixture of step (a). In some embodiments, step (b) comprises heating the mixture of step (a) to a temperature of from about 50° C. to about 100° C., for from about 30 minutes to about 3 hours. In some embodiments, step (b) comprising heating the mixture of step (a) to a temperature of about 75° C. for from about 30 minutes to about 2 hours.

Also disclosed and claimed herein is a mixture comprising, in combination, poacic acid (X) and poacidiene (Y), made by the method disclosed herein.

Also disclosed and claimed herein is a method of inhibiting fungal growth, comprising contacting a fungus with an inhibitory amount of the mixture of poacic acid (X) and poacidiene (Y) as disclosed herein.

The objects and advantages of the disclosure will appear more fully from the following detailed description of the preferred embodiment of the disclosure made in conjunction with the accompanying drawings.

### BRIEF DESCRIPTION OF THE DRAWINGS

FIG. 1 presents structures of dehydrodimers from FA and their decarboxylated products.

FIG. 2 is a partial ¹H NMR spectrum of crude products from an exemplary FA coupling reaction, showing the methoxy region. Most dimeric products have two separated peaks whereas compound C and I have only one peak due to their symmetrical structures. Minor peak overlap occurred with compounds E and D, and H, B, and H′ (an isomer of H).

FIG. 3 is a reaction scheme of decarboxylation of hydroxymatairesinol and 8-8-lactone H to generate compound K and poacidiene Y under alkaline conditions.

FIG. 4 is a partial ¹H NMR (methoxy region) of decarboxylation products from an exemplary FA coupling reaction, showing poacic acid X and poacidiene Y as major components.

FIG. 5 is a series of photographs of cultured fungus exposed to the anti-fungal composition of matter disclosed and claimed herein. The top series of photographs is of cultured Clarireedia jacksonii (ATCC 10943); the bottom series of photographs is of cultured Fusarium oxysporum f.sp. cubense tropical Race 4 (ATCC 76255). The far left photos are controls—the two strains cultured in PDA alone. Then moving to the right, exposed to DMSO, poacic acid (X), poacidiene (Y), and mixtures of X and Y. (See Examples for full details.)

FIG. 6 is a histogram showing the colony areas in the top series of photographs in FIG. 5 (i.e., for the Clarireedia jacksonii cultures).

FIG. 7 is a histogram showing the colony area in the bottom series of photographs in FIG. 5 (i.e., for the Fusarium oxysporum f.sp. cubense TR4 cultures).

FIGS. 8A-8E are photographs of cultured fungi exposed to the poacidiene/poacic acid (“PDPA”) mixture at concentrations of 250, 500, or 1000 mg/L, and histograms showing the measured colony diameters for each test. PDA plates without additives (“PDA”) and PDA plates containing DMSO served as controls. Five microorganisms were evaluated: (FIG. 8A) Alternaria solani; (FIG. 8B) Aspergillus fumigatus; (FIG. 8C) Fusarium graminearum; (FIG. 8D) Pythium ultimum; and (FIG. 8E) Sclerotinia sclerotiorum.

### DETAILED DESCRIPTION

### Abbreviations and Definitions

COSY=Correlation NMR spectroscopy. DMSO=Dimethyl sulfoxide. EtOAc=Ethyl acetate. FA=Ferulic acid (FA, F). HMBC=Heteronuclear Multiple-Bond Correlation NMR spectroscopy. HOAc=Acetic acid. HSQC=Heteronuclear Single-Quantum Coherence NMR spectroscopy. I.S.=Internal standard. NMR=Nuclear magnetic resonance spectroscopy.

As used herein, the singular forms “a,” “an,” and “the” include plural referents unless the content clearly dictates otherwise.

As used herein, the term “or” is an inclusive “or” operator and is equivalent to the term “and/or” unless the context clearly dictates otherwise.

Numerical ranges as used herein are intended to include every number and subset of numbers contained within that range, whether specifically disclosed or not. Further, these numerical ranges should be construed as providing support for a claim directed to any number or subset of numbers in that range. For example, a disclosure of from 1 to 10 should be construed as supporting a range of from 2 to 8, from 3 to 7, from 5 to 6, from 1 to 9, from 3.6 to 4.6, from 3.5 to 9.9, and so forth.

All patents, patent publications, and peer-reviewed publications (i.e., “references”) cited herein are expressly incorporated by reference to the same extent as if each individual reference were specifically and individually indicated as being incorporated by reference. In case of conflict between the present disclosure and the incorporated references, the present disclosure controls.

The elements and method steps described herein can be used in any combination whether explicitly described or not.

All combinations of method steps disclosed herein can be performed in any order, unless otherwise specified or clearly implied to the contrary by the context in which the referenced combination is made.

The method disclosed herein can comprise, consist of, or consist essentially of the essential steps and elements described herein, as well as any additional or optional elements, components, or limitations described herein or otherwise useful in synthetic organic chemistry. The disclosure provided herein suitably may be practiced in the absence of any element which is not specifically disclosed herein.

It is understood that the disclosure is not confined to the particular ingredients, compositions of matter, or steps herein illustrated and described, but embraces such modified forms thereof as come within the scope of the claims.

### Mixture of Poacic Acid (X) and Poacidiene (Y) and Methods of Production:

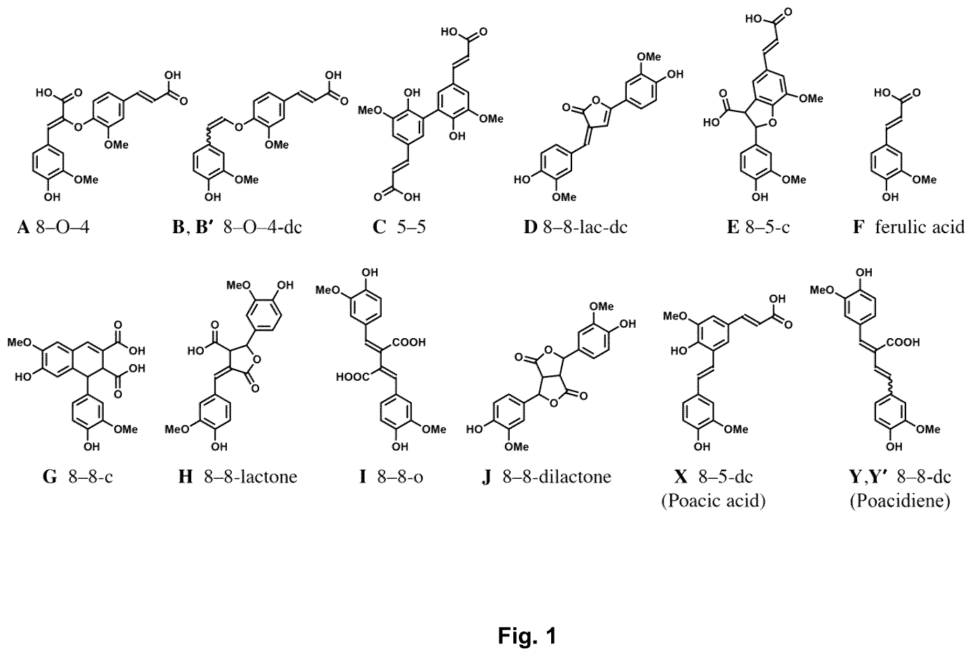

In the present disclosure, it is discovered that coupling of ferulic acid (F; FIG. 1) was regioselective under certain conditions, producing primarily the 8-5-coupled and/or 8-8-coupled dimeric products. Thus, disclosed herein is a method of fabricating the 8-5-coupled and/or 8-8-coupled products and directly decarboxylating them to produce poacic acid (X) and poacidiene (Y). It has also been found that poacic acid and poacidiene have synergistic antifungal activity when applied together, in combination. While not being limited to any particular mechanism or underlying biological phenomenon, it is thought that that the synergistic antifungal activity is due to the different modes of action between poacic acid and poacidiene via which they inhibit the growth of pathogenic fungi.

There is considerable utility and market value in a method to make a mixture of poacic acid and poacidiene without needing to isolate the two compounds from one another. The method disclosed herein thus provides a practical and economically favorable synthetic route to a very promising antifungal formulation.

Thus, disclosed herein is a method to produce a mixture comprising poacic acid (X) and poacidiene (Y) from ferulic acid (F) in a convenient and green manner, in which water is the only solvent used and no chromatography is involved. Antifungal activities of the produced mixture have been evaluated against various pathogenic fungi. The mixture was also applied to turfgrass for controlling diseases such as dollar spot and snow molds.

Specifically, disclosed herein is a method to produce a mixture comprising poacic acid (X) and poacidiene (Y) from ferulic acid (F). The method comprises subjecting a solution of ferulic acid to a free-radical coupling reaction to yield an intermediate mixture comprising ferulic acid dehydrodimers. The intermediate mixture is then decarboxylated to yield a product mixture comprising poacic acid (X) and poacidiene (Y). Preferably, the free-radical coupling reaction and the decarboxylation step are conducted in a single reaction vessel. Also, it is preferred that the two steps (free-radical coupling and decarboxylation) are conducted without any intervening processing steps. Preferably, both steps are conducted in an aqueous reaction solution.

Decarboxylating the intermediate mixture may be accomplished by heating the intermediate mixture. For example, heating the intermediate mixture to a temperature of from about 50° C. to about 100° C., for from about 30 minutes to about 3 hours; or heating the intermediate mixture to a temperature of about 75° C. for from about 30 minutes to about 2 hours.

Optionally, the method may include a third step, namely, acidifying the product mixture and extracting the product mixture with ethyl acetate. Preferably this is done at a pH of from about 1.0 to about 4.5, more preferably from a pH of from about 1.0 to about 4, and more preferably still at a pH of from about 1.0 to about 3.0.

Alternatively, the product mixture may be acidified to precipitate a mixture comprising poacic acid (X) and poacidiene (Y). The product mix can then be filtered from the reaction solution. Preferably, the solution is acidified to a pH of about 1.0 to about 4.15.

### EXAMPLES

The following examples are included solely to provide a more complete disclosure of the compositions and methods claimed herein. The examples are not intended to limit the scope of the claims in any fashion.

### Materials:

trans-Ferulic acid 1, a white to off-white crystalline powder, was purchased from Chem-Impex International Inc. (Wood Dale, Illinois, USA). All other chemicals and solvents were purchased from Aldrich (Milwaukee, Wisconsin, USA) and used as supplied.

### Methods:

### NMR Characterization and Compositional Analysis:

NMR spectra were acquired on a Bruker Biospin (Billerica, Massachusetts, USA) AVANCE 500-brand (500 MHz) spectrometer fitted with a cryogenically-cooled 5 mm Triple Resonance Inverse (TCI) gradient probe with inverse geometry (proton coils closest to the sample). Spectra were processed using Bruker's Topspin 4.4 software. Standard Bruker implementations of one- and two-dimensional (gradient-selected COSY, HSQC and HMBC) NMR experiments were used for routine structural assignments of all synthesized compounds. The conditions used for all samples were 5-10 mg in 0.5 mL acetone-d₆, with the central solvent peak (δ_{H}/δ_{C}, 2.04/29.80) used as an internal reference.

Molar percentages of each compound in the crude mixture were determined with ¹H NMR by integrating the well-resolved methoxy-peak areas from 4.02 ppm to 3.62 ppm from the individual compounds. The molar percentage of a compound was calculated from:

$$Percentage ( \% ) = 100 \times {A}_{i} / {A}_{t}$$

Where Aᵢ is the peak area for individual compound; Aₜ is the total integration of peaks from 4.02 to 3.62 ppm.

The weight percentage of poacic acid and poacidiene in the decarboxylated crude products was determined by ¹H NMR using 4-hydroxybenzaldehyde (molecular weight 122) as internal standard (I.S.). A given weight of I.S. was combined with 40 mg of sample dissolved in 5 mL acetone. Aliquots (1.5 mL) of this solution were evaporated under reduced pressure. The residues were dissolved in 0.4 mL acetone-do for NMR analysis as described above. From the ¹H NMR of the sample, integration of the aldehyde peak (from I.S.) and integration of methoxy peaks from poacic acid and poacidiene were used to calculate the purity (weight %) of the crude sample using the following equation:

$$Purity \% = [ 342 \times ( A x + A y ) \times Wis ] / ( 3 \times 122 \times Ais \times Ws ) \times 100$$

Where Ax is the peak area of one methoxyl of compound X;
Ay is the peak area of one methoxyl of compound Y;
Wis is the weight of I.S. and Ais is the peak area of aldehyde (I.S.);
Ws is the weight of sample;
342=molecular weight of compound X or compound Y;
122=molecular weight of I.S.;
3=factor recognizing that each methoxyl has three protons.

### Coupling Reactions of Ferulic Acid in Various Buffers:

A 0.1 mM solution of ferulic acid (FA) was made in pH 5.8 NaOH. A 9 mL aliquot (0.9 mmole) of this FA solution was added into a 25 mL vial containing 10 mL 0.2 M NaOH-phosphate buffer (pH 5.8, 6.4, 7.0 and 7.8) and 1 mL H₂O₂-urea (1.12 eq.). Peroxidase (type I, from horseradish, 0.25 mg) in 0.25 mL water was added. The above solution was stirred at room temperature for 1 h. The reaction products were recovered by ethyl acetate extraction (20 mL×2) after acidification to pH<3 by using 1 M HCl solution. The combined ethyl acetate solution was dried over anhydrous MgSO₄ and filtered. After evaporating the solvent under reduced pressure on a rotary evaporator, the crude products were dissolved in acetone-de and characterized by NMR. The conversion rate of FA in each reaction was calculated from ¹H NMR of the crude product.

Coupling Reaction of Ferulic Acid with Various Amounts of Peroxidase:

To 10 mL of the above FA solution was added H₂O₂-urea (1.2 eq.). Peroxidase at a concentration of 1 mg/mL was added in various amounts (0.25 mg, 0.5 mg, 0.75 mg, and 1 mg) to determine an optimal condition. Each solution was stirred at room temperature for 1 h. The reaction products were recovered and characterized by NMR as described above.

Coupling Reaction of Ferulic Acid with Various Amounts of H₂O₂:

To 10 mL of the above FA solution was added given amount (1.10, 1.15, 1.20 and 1.25 eq.) of H₂O₂-urea. Peroxidase (0.25 mg) dissolved in 0.25 mL water was added. The solution was stirred at room temperature for 1 h. The reaction products were recovered and characterized by NMR, as described above.

### Scaled-Up Dimerization of Ferulic Acid at Controlled pH:

FA (9.7 g, 50 mmol) was dissolved in 600 mL aqueous NaOH (2.0 g). The pH of the solution was adjusted with 20% v/v HOAc solution to pH 5.5. Additional water was added to make the total volume of the solution up to 800 mL. Peroxidase (8 mg) was added. H₂O₂-Urea (2.8 g, 1.15 eq) dissolved in 100 mL water was added slowly over 20 min while maintaining the pH of the reaction between 6.5 and 7.0 by adding dilute acetic acid solution successively. The reaction mixture was stirred for another 30 min after completing the addition of H₂O₂-urea solution. The final pH of the solution was 6.88. To check the reaction progress, an aliquot (2 mL) of the solution was acidified with HCl and extracted with EtOAc. The recovered products were examined by NMR, demonstrating a 95.6% conversion of ferulic acid.

The reaction was repeated with FA solution having pH of 6.5. The pH of reaction media was maintained between 6.8-7.2 during addition of H₂O₂-urea solution. The FA conversion was 94.7%, measured by NMR.

The reaction was repeated with FA solution having pH of 7.0. The pH of reaction media was maintained between 7.0 and 7.5 during addition of H₂O₂-urea solution. The FA conversion was 93.8%, measured by NMR.

The reaction was repeated with FA solution having pH of 6.1. The pH of reaction media was maintained between 7.6 and 8.0 during addition of H₂O₂-urea solution. The FA conversion was 93%, measured by NMR.

Scaled-Up Dimerization of Ferulic Acid without pH Control:

FA (50 g, 258 mmol) was suspended and stirred in 800 mL aqueous NaOH (10.31 g) solution for 3 h to dissolve all of the FA. The pH of this solution was 7.17. H₂O₂-Urea (15.15 g, 1.25 eq) was added, followed by adding 43 mg peroxidase dissolved in 15 mL water. The reaction mixture was stirred for another 40 min. The final pH of the solution was up to 8.65. An aliquot (1 mL) of the solution was acidified with HCl and extracted with EtOAc. The recovered products were examined by NMR, showing a 97.0% conversion of ferulic acid.

FA (50 g, 258 mmol) was suspended and stirred in 800 mL aqueous NaOH (10.31 g) solution for 3 h. The pH (7.2) of this solution was adjusted to 6.34 by adding dilute HOAc solution. Peroxidase, 40 mg dissolved in 10 mL water, was added, followed by slowly adding H₂O₂-Urea (14.6 g, 1.20 eq.) dissolved in 70 mL water over 20 min. The reaction mixture was stirred for another 40 min. The final pH of the solution was up to 8.56. An aliquot (1 mL) of the solution was acidified with HCl and extracted with EtOAc. The recovered products were examined by NMR, showing a 95.5% conversion of ferulic acid.

FA (50 g, 258 mmol) was suspended and stirred in 800 mL aqueous NaOH (10.8 g) solution for 3 h. The pH (8.1) of this solution was adjusted to 6.20 by adding diluted HOAc solution. H₂O₂-Urea (14.6 g, 1.20 eq) were added, followed by adding 35 mg peroxidase dissolved in 10 mL water. The reaction mixture was stirred for 60 min. The final pH of the solution was up to 8.53. An aliquot (1 mL) of the solution was acidified with HCl and extracted with EtOAc. The recovered products were examined by NMR, showing a 95.3% conversion of ferulic acid.

### Decarboxylation of the FA-Coupling Products:

As both 8-5-c E and 8-8-lactone H (8-8-lac) are readily decarboxylated under mild alkaline conditions and the resultant pH of the reaction solution following the coupling was around 8.5 (which is good for decarboxylation), the only variable considered here was the reaction time at 75° C. This is the same temperature used previously for decarboxylation of compounds similar to the 8-8-lactone. (Eklund, et al. J. Org. Chem. 2002, 67 (21), 7544-7546.)

The FA coupling products in the resultant solution under conditions described as below, initial pH of the FA solution was 6.4; 1.25 eq. H₂O₂-urea and 200 μg per mmol FA were used. After 1 h reaction, the pH of the product solution was 8.6. An aliquot of the solution was transferred into a vial and heated in a water-bath at 75° C. for various 0.5, 1, and 2 h. The solution was cooled to room temperature and acidified with HCl (pH<2) and extracted with EtOAc. After removing the solvent (EtOAc), the residual products were characterized by ¹H NMR.

### Generalized One-Pot Procedure for Production of a Poacic Acid/Poacidiene Mixture:

NaOH (11 g) was dissolved in 800 mL water, to which 50 g of FA were added. The suspension was stirred for about 3 h to completely dissolve the FA. The pH (8.3) of this solution was adjusted to 6.0 by adding dilute HOAc solution. H₂O₂-Urea (14.6 g, 1.2 eq) were added, followed by adding 32 mg peroxidase dissolved in 10 mL water. The reaction mixture was stirred for 45 min. The resultant solution in a round-bottom flask was heated in a water bath at 75° C. When the temperature of the solution reached 75° C., it was held at that temperature for 60 min. The solution was transferred into a 3 L plastic beaker and cooled down to room temperature by adding 500 g crushed ice. Then the solution was acidified by adding acetic acid or dilute HCl (1 M) to decrease the pH of the solution to about 3-4. The precipitated products were recovered by filtration through a Buchner funnel with No. 2 Whatman filter paper. The solid products were suspended and stirred in 500 mL acidic water (1% acetic acid or 0.01 M HCl) for 1 h. The mixture was filtered through a Buchner funnel with No. 2 Whatman filter paper. The recovered solid was dried under vacuum to give orange-colored products (37.0 g, 74% from FA). The purity (percentage of poacic acid plus poacidiene in the mixture) of the product was 44-50%.

### Testing Anti-Fungal Activity:

In a first series of tests, actively growing potato dextrose agar (PDA) plugs of Clarireedia jacksonii (ATCC 10943, deposited under obsolete name Sclerotinia homoeocarpa; see Salgado-Salazar et al., Fungal Biology 2018, 122 (8), 761-773) and Fusarium oxysporum f.sp. cubense tropical Race 4 (ATCC 76255) were inoculated on PDA plates supplemented with compounds of interest (8-5-DC (X), 8-8-DC (Y), the 8-5-DC (X) and 8-8-DC (Y) mixture at 1:0.6 (v/v), the 8-5-DC (X) and 8-8-DC (Y) mixture at 1:0.6 (v/v) with 40 vol % purity in water) at a concentration of 250 mg/L. PDA plates without additives and PDA plates containing DMSO were included as controls. Pictures of plates were recorded at 3 days post-inoculation (dpi) for C. jacksonii, and 5 dpi for TR4. Statistical analysis used a one-way ANOVA test (p<0.05).

In a second series of tests, fungal inhibition assays were performed on PDA plates (Becton, Dickinson and Co., 100×15 mm) containing 0, 250, 500, or 1000 mg/L poacidiene/poacic acid (“PDPA”) mixture. This mixture has poor solubility in water, so stock solutions were prepared by dissolving in dimethyl sulfoxide (DMSO) before dilution into PDA at the indicated concentrations. DMSO was added to control plates to account for its potential effect on fungal growth. Three replicates were used for each treatment. Plates were inoculated with actively growing, 5 mm-diameter plugs of Sclerotinia sclerotiorum, Fusarium graminearum, Alternaria solani, Pythium ultimum, or a spore solution of Aspergillus fumigatus, and grown at 25° C. for 2 and 7 days, respectively. From initial isolation until use in this study all isolates were stored at −80° C. on dried filter paper. Immediately upon taking out of −80° C. storage the isolates were placed on PDA and grown for up to 1 week at 25° C. before subculturing onto the amended media. Colony growth area was measured and compared to the DMSO control. All experiments were carried out in triplicate and statistical analysis used the one-way analysis of variance (ANOVA) test in GRAPHPAD PRISM software.

### Results and Discussion:

### Oxidative Coupling of FA:

Free-radical coupling FA under various conditions has been performed and studied for different purposes. The early investigation by Cartwright and Haworth (J. Chem. Soc. 1944, 535-537) involved oxidation of FA with FeCl₃ or ammonium persulphate to produce FA dilactone J (FIG. 1) in about 20% yield:

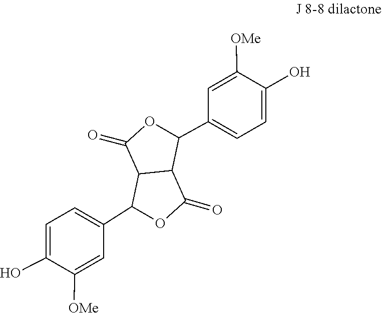 [SMILES: COc1cc(C2OC(=O)C3C(c4ccc(O)c(OC)c4)OC(=O)C23)ccc1O]

Oxidative coupling of FA catalyzed by peroxidase in acidic buffer, performed by Ward et al. (J. Biol. Chem. 2001, 276 (22), 18734-18741) produced several dehydrodimers resulting from 8-5- and 8-8-coupling of FA, of which FA dilactone was the major product. Laccase-catalyzed oxidation of FA in organic solvent/water biphasic system also produced 8-8-coupled FA dilactone J (8-8-dilactone) as a major dehydrodimer in addition to a very minor product, 8-5-c diferulic acid E (see FIG. 1 and below):

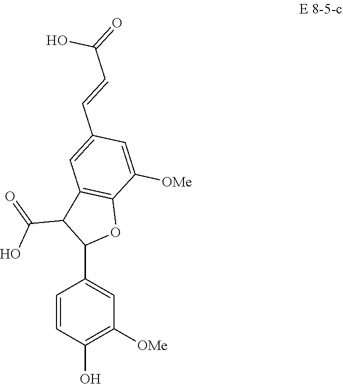 [SMILES: COc1cc(C2Oc3c(OC)cc(/C=C/C(=O)O)cc3C2C(=O)O)ccc1O]

Dimerization of FA in the peroxidase-H₂O₂ system has been shown to proceed in a regioselective manner, if quaternary ammonium (hydroxide or chloride) surfactants are used, to produce either FA dilactone J or 8-5-c E and 8-8-lactone H (two isomers) as major dimeric products (FIG. 1 and below):

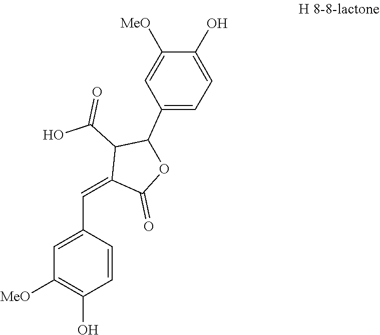 [SMILES: COc1cc(/C=C2\C(=O)OC(c3ccc(O)c(OC)c3)C2C(=O)O)ccc1O]

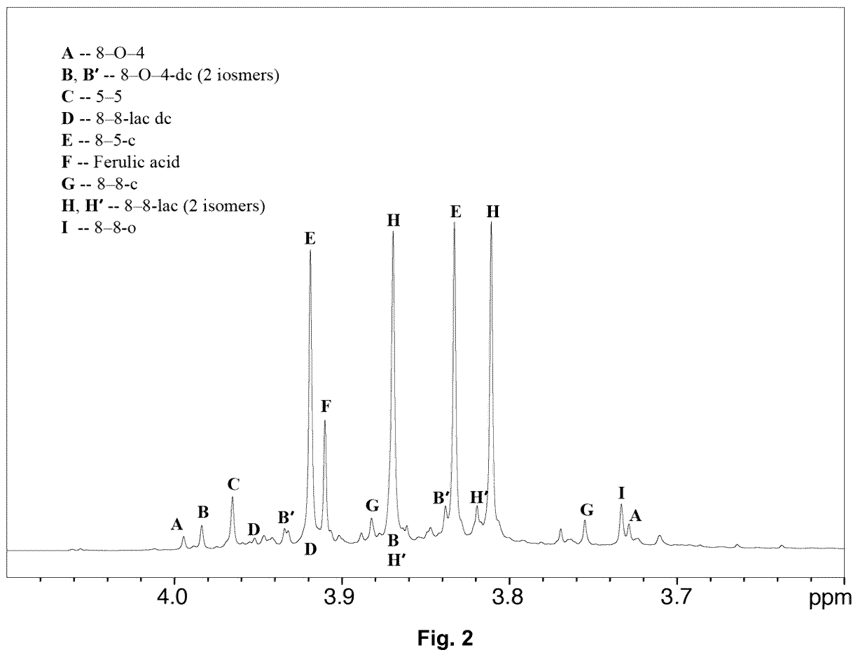

However, without surfactant added, and under acetate buffer (pH 7.5) conditions, peroxidase-catalyzed oxidation of FA also resulted in 8-5-c and 8-8-lactone as major dehydrodimers, similar to those obtained when quaternary ammonium hydroxide was used and in which the pH of the micellar solution was 7-8. It seems that the pH of the reaction medium for the FA coupling reaction plays a key role in how the dimerization products can be generated in such a system. As mentioned above, free-radical coupling of FA in acidic (pH 3-5) buffer or non-buffered aqueous organic solvent (ethanol or acetone) always produced FA dilactone J as a major dehydrodimer; it can be isolated in 23-27% yield. Here, the FA coupling reaction was performed under slightly basic conditions, with or without buffers. The resultant products were characterized by ¹H NMR. See FIG. 2, which is a partial ¹H NMR spectrum of crude products from FA coupling reaction, showing the methoxy region. Identification of the various dimeric products was by comparing chemical shifts of their characteristic methoxy groups with those of the standard/isolated compounds.

### Optimization of Coupling Conditions:

As shown in FIG. 1, the major dimeric products are diferulic acids E (8-5-c) and H (8-8-lactone) although the other minor products were also formed when the FA coupling reaction was performed under slightly basic aqueous conditions. Free-radical coupling of FA is well known to produce complex mixtures leading to oligomers and even polymers under certain conditions. Here, it is desirable to optimize conditions to produce maximal amounts of the target compounds while minimizing other products including oligomers and polymers. To do so, several parameters including pH of the reaction medium, dosage of oxidant (H₂O₂-urea), and level of peroxidase were evaluated. The composition or molar percentages of each identified compound in the product mixture were determined by integrating peak areas of each clean peak belonging to individual dimers. The whole sum from integrating all the methoxy peaks between shifts 4.02 and 3.62 ppm was used as a measure of the total molar equivalents of product compounds. The calculated percentages of individual products A-I from FA coupling reactions are summarized in Table 1. In phosphate buffer (pH 5.8 to 7.8) the major dimeric products are 8-5-coupled diferulic acid E comprising 25-28% of the total product, and 8-8-coupled FA lactone H comprising 26-33% of the crude mixture. About 10% of FA dilactone J was detected in the coupling product mixture when the reaction was conducted in pH 5.8 buffer. Although the ratios between compounds E and H were quite consistent when the coupling reactions were carried out in buffer with pHs ranging from 6.4 to 7.8, the ratios of compound I (8-8-0), A, and B gradually increased with increasing pH. There is a slight advantage when the reaction was performed in neutral (pH 7) condition at which the combined yields of E and H as well as total dimers were maximized. Considering the dosage of H₂O₂-urea and peroxidase, no significant differences were found under conditions used here for FA coupling reactions in terms of yields of compounds E and H as well as their ratios. However, the use of 1.25 eq. H₂O₂-urea, i.e., an excess of oxidant, could result in formation of more oligomers or polymer, leading to lower amounts of E and H as well as total dimeric products.

TABLE 1
Molar percentages of products A-I from ferulic acid coupling reactions.

| | | | | | | | | | | | Total |
|---|---|---|---|---|---|---|---|---|---|---|---|
| | | E | H | E + H | A | B | C | D | G | I | dimers |
| pH | 5.8 | 25.8 | 25.8 | 51.6 | 0.1 | 1.7 | Tr. | 2.6 | 1.8 | 0.7 | 68.3* |
| buffer ^{a} | 6.4 | 25.0 | 32.8 | 57.8 | 0.1 | 2.8 | Tr. | 3.6 | 2.1 | 1.0 | 66.5 |
| | 7.0 | 28.0 | 33.0 | 61.0 | 0.3 | 2.6 | 0.1 | 3.7 | 1.7 | 1.2 | 70.7 |
| | 7.8 | 26.2 | 30.1 | 56.3 | 0.9 | 5.4 | 1.1 | 3.0 | 1.6 | 1.7 | 70.0 |
| H₂O₂-urea ^{b} | 1.10 | 25.3 | 28.0 | 53.3 | 1.2 | 5.6 | 1.7 | 2.5 | 1.9 | 2.0 | 68.2 |
| (eq.) | 1.15 | 25.3 | 27.9 | 53.2 | 1.5 | 5.0 | 1.5 | 2.4 | 2.4 | 2.0 | 68.1 |
| | 1.20 | 25.8 | 28.7 | 54.5 | 1.2 | 5.3 | 1.4 | 2.5 | 2.5 | 2.1 | 69.5 |
| | 1.25 | 25.2 | 24.8 | 50.0 | 1.0 | 5.0 | 1.2 | 2.9 | 2.7 | 1.0 | 63.8 |
| Peroxidase ^{c} | 125 | 24.3 | 29.2 | 53.5 | 1.4 | 5.7 | 2.1 | 1.5 | 2.1 | 1.7 | 68.0 |
| (μg/mmol | 250 | 24.2 | 30.5 | 54.8 | 0.7 | 6.5 | 2.2 | 1.8 | 2.5 | 1.6 | 70.0 |
| FA) | 375 | 24.0 | 30.4 | 54.4 | 1.4 | 6.9 | 2.2 | 1.9 | 2.2 | 1.5 | 68.3 |
| | 500 | 24.3 | 29.9 | 54.2 | 1.6 | 7.0 | 2.1 | 2.3 | 2.3 | 1.5 | 71.0 |

*9.8% of FA dilactone (J) were produced under this condition.
^{a} 1.12 eq. H₂O₂-urea, 0.25 mg peroxidase/mmol FA, 1 h reaction time.
^{b} pH of FA solution was 5.8; 0.25 mg peroxidase/mmol FA; 1 h reaction time.
^{c} pH of FA solution was 6.0; 1.2 eq. H₂O₂-urea was used; 1 h reaction time.

It is common for enzyme reactions to be performed in biological buffer solutions. However, from a practical point of view for large-scale production of chemicals, it is preferable to avoid using huge amounts of inorganic salts that may generate extra cost for downstream processes. Thus, a series of FA coupling reactions was performed in aqueous solution without buffer; the pH of the reaction solution was controlled to within given ranges by adding dilute acetic acid while the H₂O₂-urea solution is gradually introduced into the FA solution containing peroxidase. The results of NMR analysis of the crude products are listed in Table 2 in which it is evident that controlling pH of the reaction to a narrow range generates comparable results to those found under buffered conditions with similar pHs. It is interesting to note that FA coupling reactions performed in solutions with pH ranges from 6.5 to 7.2 generated results consistent with those obtained from the FA coupling reaction carried out in pH 7.0 buffer.

TABLE 2
Molar percentages of products A-I from scale-up coupling reactions of FA.

| | Starting | pH | | | E + | | | | | | | Total |
|---|---|---|---|---|---|---|---|---|---|---|---|---|
| | pH | range | E | H | H | A | B | C | D | G | I | dimers |
| pH ^{a} | 5.5 | 6.5-7.0 | 28.4 | 34.2 | 62.6 | 0.9 | 2.2 | 0.3 | 1.8 | 1.6 | 1.4 | 70.8 |
| control | 6.5 | 6.8-7.2 | 28.5 | 35.0 | 63.5 | 1.0 | 2.3 | 0.2 | 1.7 | Tr. | 1.3 | 70.0 |
| | 7.0 | 7.0-7.5 | 28.0 | 32.1 | 60.1 | 0.3 | 2.5 | 0.3 | 1.0 | 1.3 | 1.6 | 67.3 |
| | 6.1 | 7.6-8.0 | 27.4 | 27.5 | 54.9 | 0.4 | 2.5 | 1.2 | 1.2 | 2.2 | 1.7 | 64.1 |
| No pH | 6.20 ^{b} | → 8.53 | 26.0 | 28.5 | 54.5 | 1.2 | 3.0 | 2.3 | 0.6 | 3.5 | 3.0 | 67.7 |
| control | 6.34 ^{c} | → 8.56 | 25.1 | 27.6 | 52.7 | 0.9 | 2.8 | 2.5 | 2.0 | 4.4 | 2.2 | 67.5 |
| | 7.17 ^{d} | → 8.65 | 25.0 | 27.3 | 52.3 | 1.4 | 3.7 | 3.0 | 1.5 | 3.4 | 2.0 | 67.3 |

^{a} 1.15 eq. H₂O₂-urea, 0.16 mg peroxidase/mmol FA.
^{b} Peroxidase (0.16 mg peroxidase/mmol FA) in water was added into FA solution having 1.20 eq. H₂O₂-urea.
^{c} 1.20 eq. H₂O₂-urea was added into FA solution containing 0.135 mg peroxidase/mmol FA.
^{d} Peroxidase (0.17 mg peroxidase/mmol FA) in water was added into FA solution having 1.25 eq. H₂O₂-urea.

Next, the FA coupling reaction was performed without pH control to determine whether the operation of FA coupling reaction could be further simplified. Several FA coupling reactions were performed in aqueous solution with various starting pHs in which the H₂O₂-urea solution was slowly added into the FA solution containing peroxidase, or peroxidase dissolved in water was added into the FA solution containing the H₂O₂-urea. The results listed in Table 2 indicate that the combined yields of compounds E and H were slightly lower than those obtained from reactions under pH control, although the total identified dimeric products were formed in higher yields apparently due to higher yields of compounds B, C, G, and I. (See FIG. 1.) It was observed that with the development of the FA coupling reaction the pH of the solution increased to ˜8.6, regardless of how H₂O₂-urea or peroxidase was added. These results indicate that the FA coupling reaction can be performed without tight pH control with little compromise to the yields of products E and H, greatly simplifying the operation.

### Decarboxylation of Dehydrodimers E and H to Poacic Acid and Poacidiene:

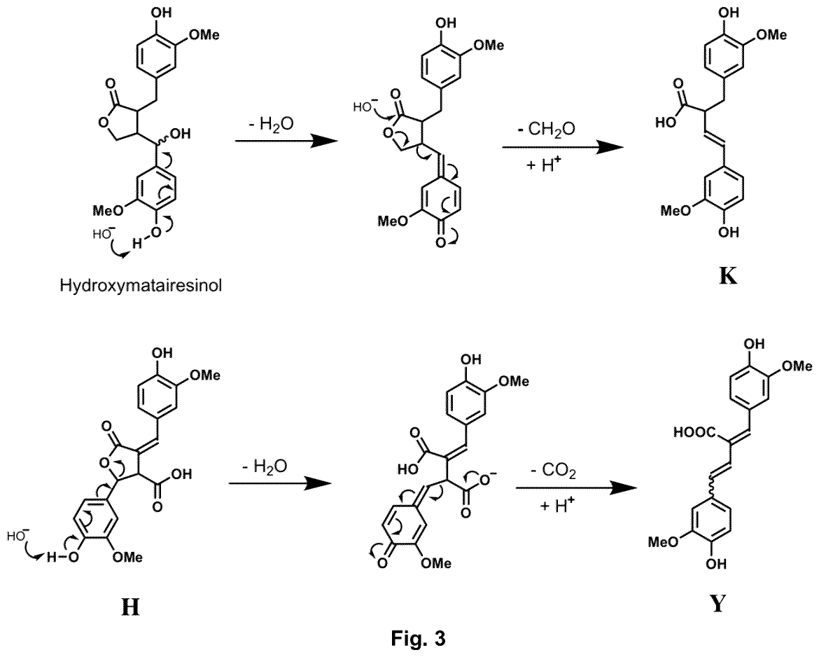

Previous efforts to scale up synthesis of 8-5dc X (poacic acid) involved several steps starting from ethyl ferulate. Hydrolysis and decarboxylation of 8-5-coupled diethyl diferulate in strong basic NaOH solution at 90° C. was the last step to produce the target compound. Although this approach can be adopted for industrial production, the overall yield was relatively low and large amounts of organic solvent (ethanol or acetone) were required to synthesize the key intermediate 8-5c diferulate. It was envisioned that compound E, 8-5-c diferulic acid, should be a better candidate than its diethyl diferulate analog because it was reported that 8-5-c diferulic acid can be converted to 8-5-dc readily under very mild condition (pH 6-7, 37° C.). It has also been shown that hydroxymatairesinol, under alkaline solution (0.6 M NaOH, 80° C.), is converted to a phenolic acid, a dihydro analog of poacidiene Y. (Eklund, et al. J. Org. Chem. 2002, 67 (21), 7544-7546.) Considering that compound H (8-8-lactone) shares a similar chemical propensity under alkaline conditions, it was conjectured that compound H should be converted to poacidiene Y under similar or even milder conditions. See FIG. 3 for the two synthetic routes, namely the decarboxylation of hydroxymatairesinol to generate compound K (top reaction) and the decarboxylation of 8-8-lactone H to yield poacidiene Y (bottom reaction). Both reactions occur under alkaline conditions.

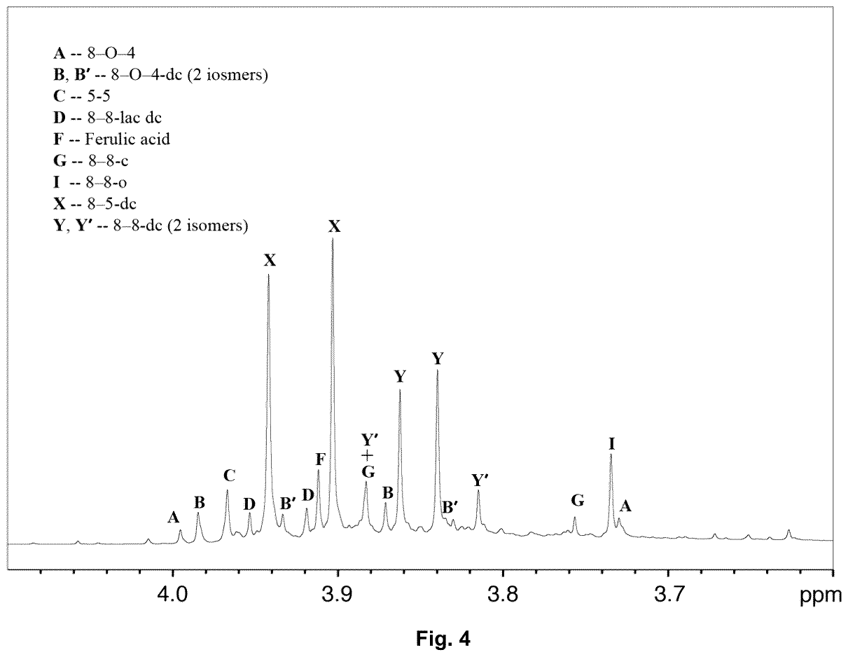

As discussed above, when the FA coupling reaction was performed without pH control, the pH of the solution increases up to 8.5-8.6. In such a pH range 8-5c diferulic acid E should be readily converted to 8-5-dc X by thermal decarboxylation. Thus, the coupling product solution was kept at 75° C. for 2 h and the decarboxylation products were extracted with ethyl acetate after acidification with acetic acid or dilute HCl. The recovered crude products were examined by ¹H NMR. See FIG. 4, which is the partial ¹H NMR (methoxy region) of the decarboxylation products from the FA coupling reaction, showing poacic acid X and poacidiene Y as the major components. The NMR result indicates that compound E and H are completely converted and the major products are poacic acid X and poacidiene Y, accompanied by other minor compounds including compounds A, B, C, D, G, and I. When treated at 75° C. in saturated NaHCO₃ solution (pH 8.7), compound E was cleanly and completely transformed into poacidiene in 45 min. However, prolonging the treatment time over 60 min produced some side-products. Based on TLC analysis of the mixtures, these side-products are in slower-moving fractions, implying that they are oligomers derived from poacidiene. Although compound H was not isolated from the thermal treatment tested, a kinetic study using crude FA coupling products indicated that compound H was degraded faster than E (results not shown) under the thermal treatment conditions. These results indicated that keeping the coupling reaction product solution (pH 8.5) at 75° C. for 60 min is sufficient to completely convert both compounds E and H into the corresponding decarboxylated products X and Y. Prolonged heating could generate more complex mixtures. Therefore, for scale up production of poacic acid and poacidiene in this one-pot two-step process, it is preferable to use 1 h heating at 75° C. for the decarboxylation.

### Recovery or Isolation of Poacic Acid and Poacidiene:

It is a common practice to use solvent extraction for recovering organic substances from aqueous solutions. However, acid-promoted precipitation techniques are a much simpler and provide a better option for scale-up production. Precipitation under acidic conditions allows partial separation of compounds with different solubilities; the less water-soluble compounds are enriched in the precipitates and the more water-soluble ones remain in solution. Poacic acid and poacidiene are decarboxylated diferulic acids. They have one less carboxylic group than the normal diferulic acids including compounds A, C, G, and I. In other words, poacic acid and poacidiene are less soluble in acidic water and can be readily enriched by acid precipitation.

TABLE 3
Molar percentages of decarboxylation products from FA dehydrodimers.

| | | | | X + | | | | | | | Total |
|---|---|---|---|---|---|---|---|---|---|---|---|
| Sample Description | | X(E) | Y(H) | Y | A | B | C | D | G | I | dimers |
| Dehydrodimers (pH 7.17) | | (25.0)* | (27.3)* | | 1.4 | 3.7 | 3.0 | 1.5 | 3.4 | 2.0 | 67.3 |
| Products | Extracted | 20.6 | 17.7 | 38.3 | 1.9 | 5.8 | 3.6 | 2.9 | 6.3 | 8.8 | 67.6 |
| | (75° C., 1.5 h) | | | | | | | | | | |
| | Precipitated | 20.8 | 17.8 | 38.6 | 1.5 | 5.5 | 3.0 | 2.6 | 4.0 | 5.0 | 60.2 |
| | (pH 1.0) | | | | | | | | | | |
| Dehydrodimers (pH 6.20) | | (26.0) | (28.5) | | 1.2 | 3.0 | 2.1 | 1.0 | 3.5 | 2.0 | 67.3 |
| Products | Extracted | 21.2 | 20.7 | 41.9 | 1.5 | 5.4 | 2.4 | 3.5 | 4.4 | 9.0 | 68.1 |
| | (75° C., 1.0 h) | | | | | | | | | | |
| | Precipitated ^{a} | 25.0 | 18.0 | 43.0 | 1.3 | 5.0 | 2.7 | 3.0 | 2.2 | 3.5 | 60.7 |
| | (pH 3.85) | | | | | | | | | | |
| Dehydrodimers (pH 6.34) | | (25.1) | (27.6) | | 0.9 | 2.8 | 2.5 | 2.0 | 4.4 | 2.2 | 67.5 |
| Products | Extracted | 20.0 | 19.4 | 39.4 | 1.0 | 3.4 | 2.6 | 3.0 | 5.6 | 8.0 | 63.0 |
| | (75° C., 2.0 h) | | | | | | | | | | |
| | Precipitated ^{b} | 27.4 | 19.6 | 47.0 | 0.8 | 3.5 | 2.9 | 3.2 | 2.4 | 3.0 | 62.8 |
| | (pH 4.15) | | | | | | | | | | |

*numbers in parentheses correspond to E or H
^{a} Crude (purity 44.2% calculated with I.S.) yield was 73% wt.
^{b} Crude (purity 50.0%) yield was 71% wt.

Table 3 lists compositional analysis results for several samples (dehydrodimers and decarboxylation products) obtained from the FA coupling reaction followed by decarboxylation. In this one-pot two-step process, the first step (free-radical coupling of FA) produces FA dehydrodimers in which the total yield of identified dimers was 67% and the combined yield of compounds E and H was 52-54%. After thermal treatment (75° C., 1-2 h), the decarboxylation products were recovered either by solvent (ethyl acetate) extraction of the acidified (pH 1.0) slurries, or by precipitation at various pHs (1.0, 3.85, or 4.15) and filtration. About 80% of compound E was converted to poacic acid X whereas about 63-70% of compound H was directed to poacidiene Y under these decarboxylation conditions. Comparing the molar percentage data obtained from samples before and after decarboxylation, it is found that about 21-27% of compound H had been converted to compounds G and I. As poacic acid and poacidiene are products of interest, the combined percentage of both compounds in crude products is the key for choosing precipitation parameters. It is apparent that under strongly acidic conditions (pH 1.0) precipitation was not able to purify or enrich the target products. The compositions of precipitated sample were very close to those of the solvent-extracted sample. However, samples obtained by precipitation at pH 4 showed an improved purity from the extracted sample (47% vs 39%). The molar percentages determined by ¹H NMR were consistent with weight percentages obtained by ¹H NMR using the internal standard (4-hydroxybenzaldehyde), suggesting that ¹H NMR analysis of these FA coupling dehydrodimers and decarboxylation products was a simple and reliable method.

### Anti-Fungal Activity of the Mixture of Poacic Acid and Poacidiene:

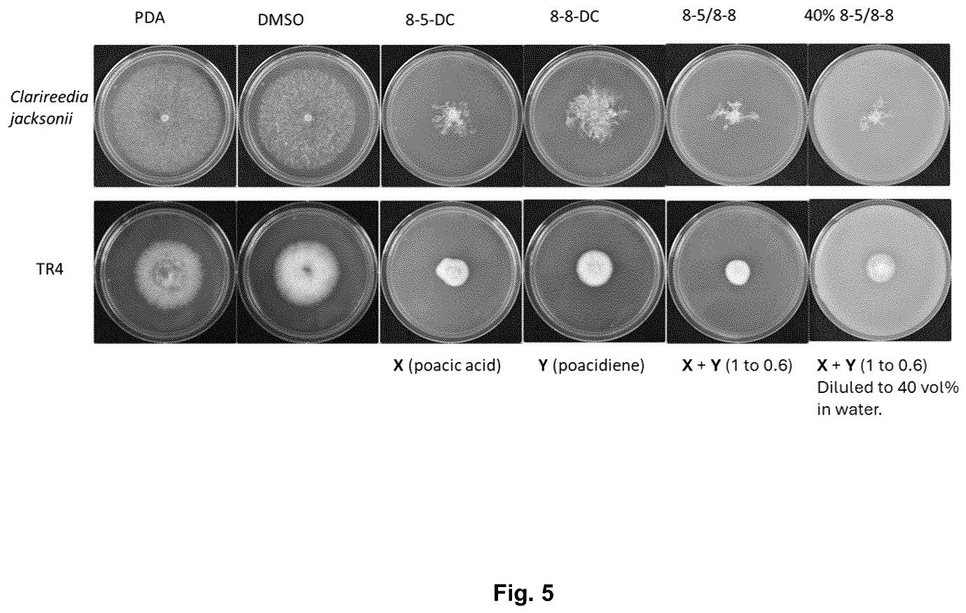

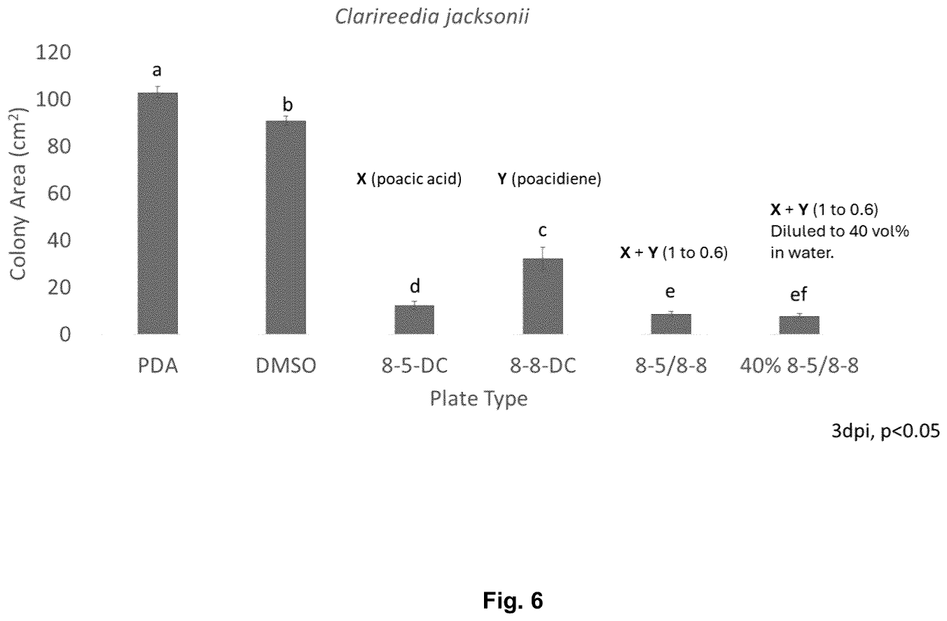

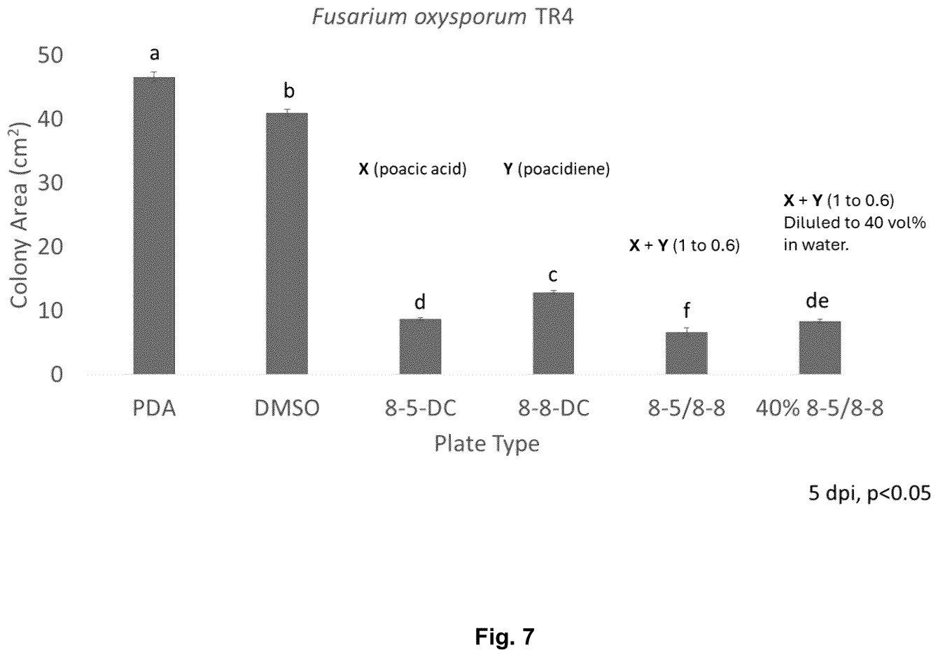

In the first series of tests, the antifungal properties of poacic acid (X), poacidiene (Y), and mixtures thereof were evaluated against Clarireedia jacksonii and Fusarium oxysporum f. sp. cubense TR4. Representative photographs of the cultured fungi are shown in FIG. 5. In each series, the far-left panels show control colonies grown on PDA alone. The adjacent panels show cultures exposed sequentially to DMSO, poacic acid (X), poacidiene (Y), and the mixtures of X+Y. As illustrated in the figure, fungal growth was substantially inhibited by the X+Y mixture in comparison to individual compounds or controls. Quantitative analysis of colony area is summarized in FIG. 6 for C. jacksonii and in FIG. 7 for F. oxysporum f. sp. cubense TR4. The histograms show that exposure to the X+Y mixtures resulted in the smallest mean colony areas among all treatments, indicating enhanced antifungal efficacy when the two compounds were combined.

Also further seen in the last two sets of panels (corresponding to the X+Y mixture at a 1:0.6 ratio, with or without water dilution), the readily prepared mixtures outperformed either of the well-established individual antifungals, even when substantially diluted. As noted above, this enhanced activity may be attributed to the two orthogonal antifungal mechanisms of the components.

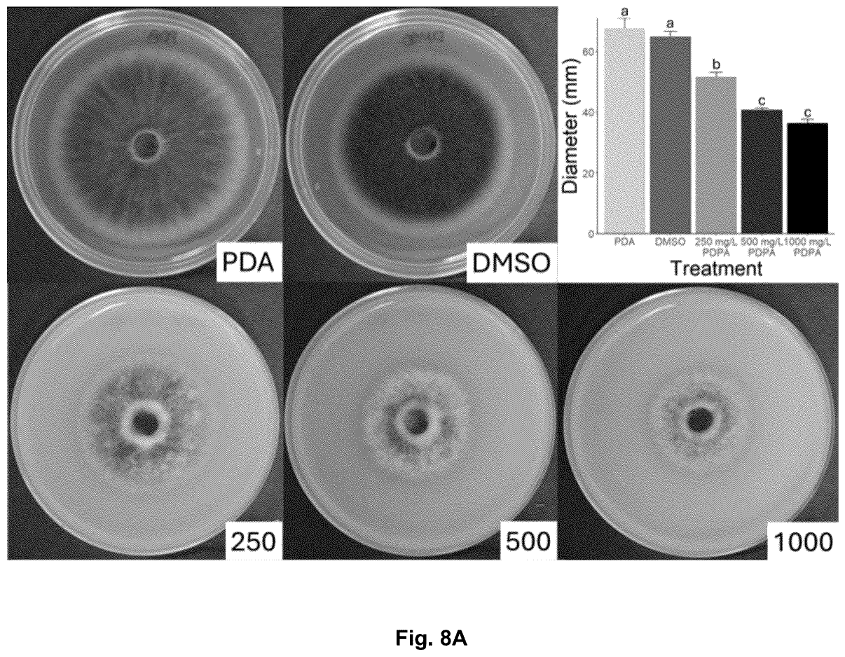

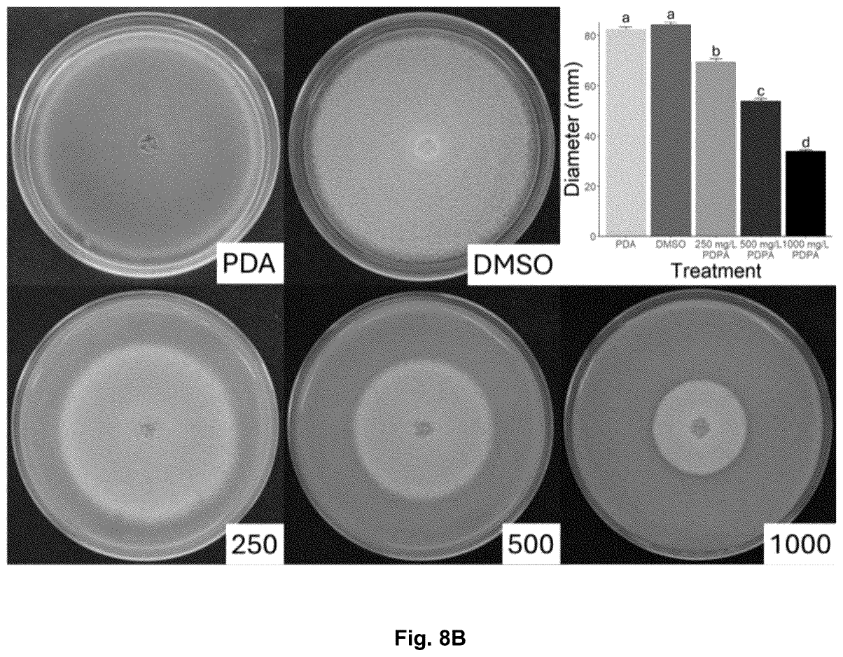

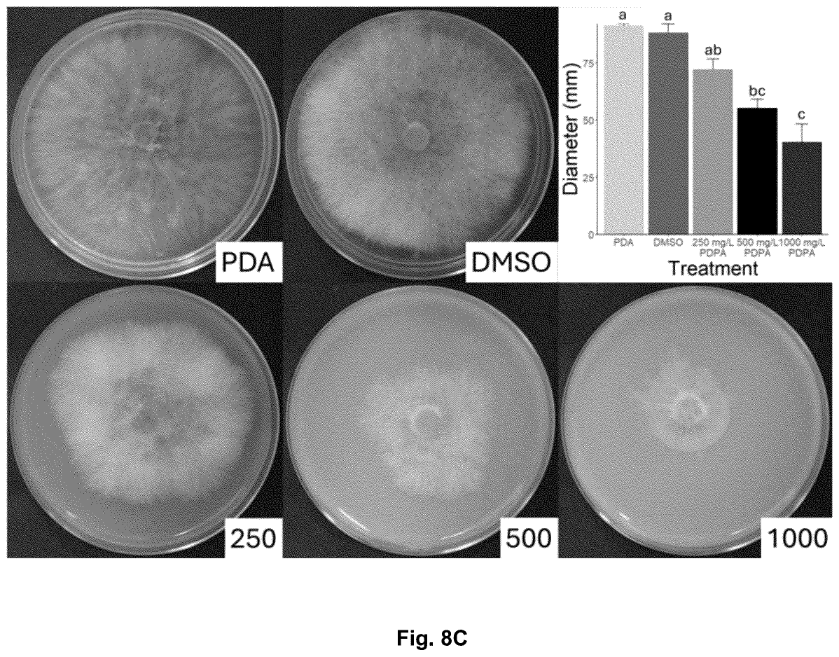

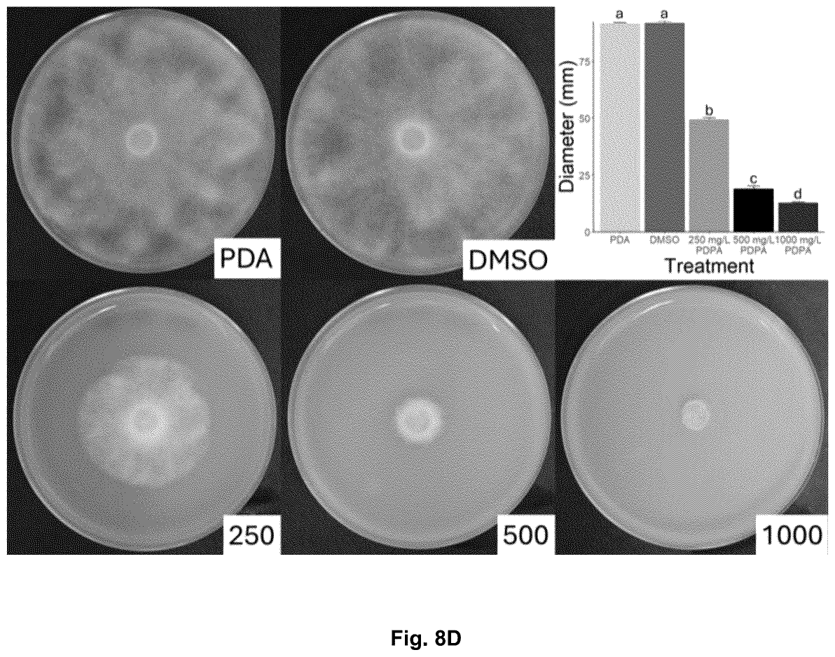

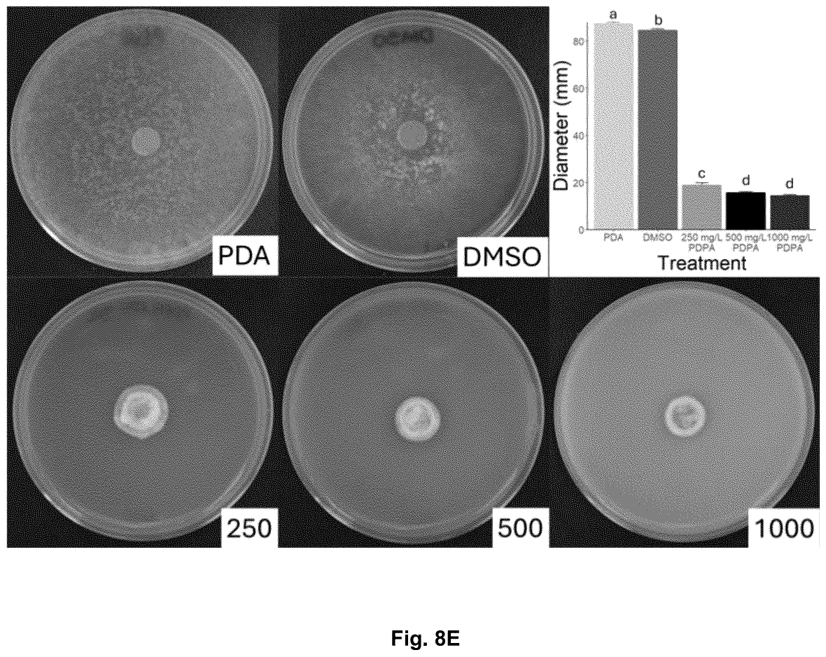

In the second series of tests, five microorganisms were used to evaluate the in vitro inhibitory activity of the poacic acid/poacidiene (“PDPA”) mixture: three common plant fungal pathogens (Sclerotinia sclerotiorum, Alternaria solani, and Fusarium graminearum), one oomycete (Pythium ultimum), and one opportunistic human pathogen (Aspergillus fumigatus). As shown in FIGS. 8A-8E, incorporation of the PDPA mixture into culture media resulted in a significant, dose-dependent reduction in growth across all tested organisms (p<0.01). Compared to the DMSO control, overall growth inhibition reached up to 83.6% for S. sclerotiorum, 56% for F. graminearum, 45.8% for A. solani, 86.4% for P. ultimum, and 59.1% for A. fumigatus. These findings indicate that the PDPA mixture exhibits strong antifungal and anti-oomycete activity.

### CONCLUSIONS

Disclosed is a facile, one-pot, two-step process for producing a mixture of poacic acid and poacidiene from ferulic acid. The product mixture obtained from this process showed excellent antifungal activity against various plant pathogenic fungi. The overall yields of the active ingredients (poacic acid and poacidiene) from ferulic acid was about 35%. The process uses ferulic acid as the raw material. Ferulic acid is an easily available and abundant natural compound. The reaction medium is water. Therefore, the method has the characteristics required for “green” production (i.e., it does not use organic solvents). It has been demonstrated that the produced antifungal mixture has improved activity due to the synergistic and mechanistically orthogonal effects of poacic acid and poacidiene. The easy operations (no high temperatures or pressures involved) and water solvent make it readily adaptable for large-scale industrial production.

What is claimed is:

1. A method to produce a mixture comprising poacic acid and poacidiene from ferulic acid, the method comprising:
a. subjecting a solution of ferulic acid to a free-radical coupling reaction to yield an intermediate mixture comprising ferulic acid dehydrodimers; and
b. decarboxylating the intermediate mixture of step (a) to yield a product mixture comprising poacic acid and poacidiene.

2. The method of claim 1, wherein steps (a) and (b) are conducted in a single reaction vessel.

3. The method of claim 2, wherein steps (a) and (b) are conducted without any intervening processing steps between steps (a) and (b).

4. The method of claim 1, wherein step (b) comprises decarboxylating the intermediate mixture of step (a) by heating the intermediate mixture of step (a).

5. The method of claim 4, wherein step (b) comprises heating the mixture of step (a) to a temperature of from about 50° C. to about 100° C., for from about 30 minutes to about 3 hours.

6. The method of claim 5, wherein step (b) comprising heating the mixture of step (a) to a temperature of about 75° C. for from about 30 minutes to about 2 hours.

7. The method of claim 1, further comprising, after step (b):
c. acidifying the product mixture of step (b) and extracting the product mixture with ethyl acetate.

8. The method of claim 7, wherein step (c) comprises acidifying the product mixture of step (b) to a pH of from about 1.0 to about 4.5.

9. The method of claim 7, wherein step (c) comprises acidifying the product mixture of step (b) to a pH of from about 1.0 to about 4.

10. The method of claim 7, wherein step (c) comprises acidifying the product mixture of step (b) to a pH of from about 1.0 to about 3.

11. The method of claim 1, further comprising, after step (b):
c. acidifying the product mixture of step (b) to precipitate a mixture comprising poacic acid and poacidiene.

12. The method of claim 11, wherein step (c) comprises acidifying the product mixture of step (b) to a pH of from about 1.0 to about 4.15.

13. The method of claim 1, wherein the solution of step (a) is an aqueous solution.

14. A method to produce a mixture comprising poacic acid and poacidiene from ferulic acid, the method comprising:
a. subjecting an aqueous ferulic acid solution to a free-radical coupling reaction to yield an intermediate mixture comprising ferulic acid dehydrodimers; and
b. decarboxylating the intermediate mixture of step (a) to yield a product mixture comprising poacic acid and poacidiene.

15. The method of claim 14, wherein steps (a) and (b) are conducted in a single reaction vessel.

16. The method of claim 15, wherein steps (a) and (b) are conducted without any intervening processing steps between steps (a) and (b).

17. The method of claim 14, wherein step (b) comprises decarboxylating the intermediate mixture of step (a) by heating the intermediate mixture of step (a).

18. The method of claim 14, wherein step (b) comprises heating the mixture of step (a) to a temperature of from about 50° C. to about 100° C., for from about 30 minutes to about 3 hours.

19. The method of claim 18, wherein step (b) comprising heating the mixture of step (a) to a temperature of about 75° C. for from about 30 minutes to about 2 hours.

20. A mixture comprising, in combination, poacic acid and poacidiene, made by a method comprising:
a. subjecting a solution of ferulic acid to a free-radical coupling reaction to yield an intermediate mixture comprising ferulic acid dehydrodimers; and
b. decarboxylating the intermediate mixture of step (a) to yield a product mixture comprising poacic acid and poacidiene.

21. A method of inhibiting fungal growth, comprising contacting a fungus with an inhibitory amount of a mixture as recited in claim 20.
